(19) United States
(12) Patent Application Publication
(10) Pub. No.: US 2003/0171570 A1
(43) Pub. Date: Sep. 11, 2003

(54) Reactive monomers for the oligonucleotide and polynucleotide synthesis , modified oligonucleotides and polynucleotides, and a method for producing the same

(76) Inventor: Markus Schweitzer, Frankfurt (DE)

Correspondence Address: Connolly Bove Lodge & Hutz, P O Box 2207, Wilmington, DE, 19899-2207, US

(21) Appl. No.: 10/221,917

(22) PCT Filed: Feb. 19, 2001
(86) PCT No.: PCT/EP01/01799

(30) Foreign Application Priority Data
Mar. 18, 2000 (DE) 10013600.1

(51) Int. Cl. C07H 21/04; C07H 19/04

(52) U.S. Cl. 536/25.32; 536/26.1

(57) ABSTRACT

The invention relates to the production of modified oligonucleotides and to their use for conjugation reactions. The invention further relates to reagents and to methods for producing aldehyde-modified oligonucleotides that contain aldehydes that are protected (masked) as acetals. Once said acetals are incorporated into the oligonucleotides the oligonucleotides are converted to aldehydes and are used for conjugation. The conjugation reaction can be carried out with the free oligonucleotide or with the oligonucleotide that is still immobilized on the substrate.

The invention relates to oligonucleotides and polynucleotides, which have been modified with at least one acetal or aldehyde group, and to a method for preparing such modified oligonucleotides and polynucleotides and the novel monomeric building blocks required therefor.

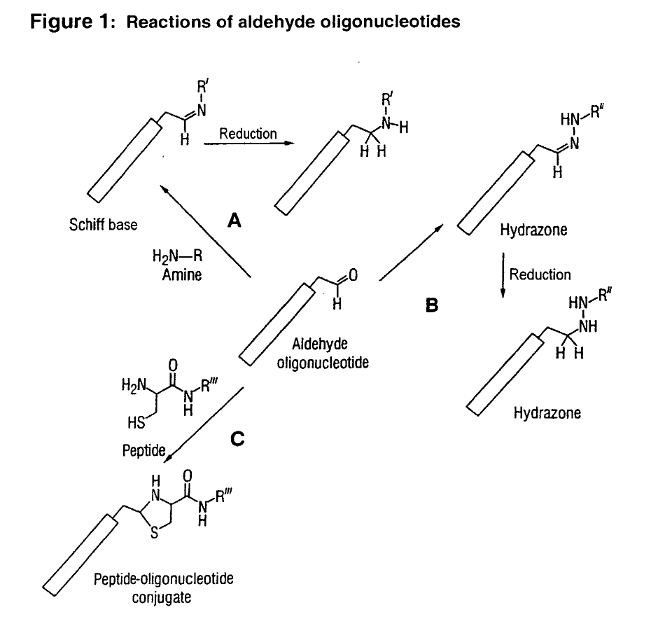

Aldehydes are reactive groups which are used for conjugating biomolecules to, for example, fluorophores, reporter groups, proteins, nucleic acids and other biomolecules, small molecules (such as biotin) or else for immobilizing biomolecules on surfaces (see, by way of example: Hermanson, G. T.; Bioconjugate Techniques, Academic Press, San Diego 1996; Timofeev, E. N.; Kochetkova, S. V.; Mirzabekov, A. D.; Florentiev, V. L., Nucleic Acids Res. 24 (1996) 3142). Since neither proteins nor nucleic acids in their natural form carry aldehydes, the latter are particularly suitable for a specific modification of the biomolecules. Carbohydrates, although aldehydes by nature, are mostly present as (cyclic) acetals or hemiacetals and, in this form, do not have the typical aldehyde reactivity either. Therefore, they can be used likewise for directed conjugations with aldehydes. Examples from the prior art of reactions of aldehydes, which can be used for conjugating biomolecules, are listed in FIG. 1, reactions A and B.

Apart from aldehydes, further reactive groups which are suitable for the conjugation of biomolecules are already known. An overview of methods for functionalizing oligonucleotides by phosphoramidite derivatives is presented in Beaucage, S. L., et al. Tetrahedron, Elsevier Science Publishers, Amsterdam, NL, Vol. 49, No. 10, 1993, pages 1925-1963. In addition, phosphonic esters as described by Bednarski, K. et al. Bioorganic & Medical Chemistry Letters, Oxford, GB, Vol. 5, No. 15, Aug. 3, 1995, pages 1741-1744 or in JP 58152029 A or phosphorylated acetals (Razumov, A. I., et al. Chemical Abstracts, Vol. 89, No. 15, Oct. 9, 1978, abstract No. 129604) have played no part so far in the introduction of aldehyde groups into oligonucleotides.

At present, different ways of introducing aldehydes into oligonucleotides are available, all of which are based on oxidation of a vicinal diol with sodium periodate to give the aldehyde or a bis-aldehyde.

First to be mentioned is the oxidation of oligonucleotides using 3′-terminal ribonucleotides (for this, see Timofeev, E. N.; Kochetkova, S. V.; Mirzabekov, A. D.; Florentiev, V. L., Nucleic Acids Res. 24 (1996) 3142; Lemaitre, M.; Bayard, B.; Lebleu, B., Proc. Natl. Acad. Sci. U.S.A. 84 (1987) 648). In this way, a ribonucleotide which forms the 3′ end of an oligonucleotide is oxidized by periodate to give a bis-aldehyde. This aldehyde then forms with amines or hydrazides cyclic adducts (morpholine structure) which can be used for conjugation.

This method has the crucial disadvantage that always a nucleotide of the 3′ end of an oligonucleotide has to be sacrificed for the conjugation. More-over, this approach does not provide the possibility of altering the distance between the oligonucleotide and the conjugation partner.

The second possibility is to couple a phosphoramidite of a protected vicinal diol to the 5′ end of an oligonucleotide (Lemaitre, M.; Bayard, B.; Lebleu, B., Proc. Natl. Acad. Sci. U.S.A. 84 (1987) 648). Here, a specifically prepared building block which carries a masked vicinal diol group is coupled to the 5′ end of an oligonucleotide. After synthesis, deprotection and working-up of the oligonucleotide, a vicinal diol group is then present, which is likewise oxidized with periodate to give the aldehyde. Such vicinal diols are likewise described in EP 0 523 078 A1.

Furthermore, the use of a modified nucleotide or nucleotide analog which carries a protected vicinal diol on a side chain for introducing an aldehyde group into an oligonucleotide is state of the art (Dechamps, M.; Sonveaux, E., Nucleosides Nucleotides 14 (1995) 867; Dechamps, M.; Sonveaux, E., Nucleosides Nucleotides 17 (1998) 697; Trevisiol, E.; Renard, A.; Defrancq, E.; Lhomme, J., Tetrahedron Lett. 38 (1997) 8687). However, this way requires a synthesis of considerable complexity.

All three ways have in common that the aldehyde must be generated by oxidizing a vicinal diol with sodium periodate. This reagent must then be removed prior to the conjugation reaction. Furthermore, this way is incompatible for molecules which carry other periodate-oxidizable groups. Thus it is impossible, for example, to specifically modify the 5′ end of an RNA strand without the 3′ end of the oligonucleotide being oxidized, too. and can be carried out easily and without great complexity starting from storage-stable reactants would be advantageous.

The object of the present invention is therefore to provide reactive monomers which are compatible with the conditions of oligonucleotide and polynucleotide synthesis and to prepare and provide modified oligo- and polynucleotides which are readily manageable and can be converted easily to their corresponding derivatives containing aldehyde groups.

The object is achieved by novel monomeric acetals and acetal-modified oligonucleotides and polynucleotides which can be stored very easily and provide easy access to aldehyde-modified oligo- and polynucleotides. In addition, the monomeric acetals of the invention and also the acetal-modified oligonucleotides and polynuleotides are stable to the conditions of the standard methods for oligo- and polynucleotide synthesis or oligo- and polynucleotide duplication, such as, for example, the phosphoramidite method or the PCR, and to the reaction conditions for introducing and removing common protective groups.

Thus the present invention relates to a reactive monomer of the formula (I), wherein l, v independently of one another are 0 or 1 and a is an integer between 1 and 5, preferably 1 to 3,

X—Lₗ—Vᵥ—(A)ₐ (I)

and wherein

X [lacuna] a reactive phosphorus-containing group for the oligonucleotide synthesis, such as, for example, a phosphoramidite (II) or such as a phosphonate (III)

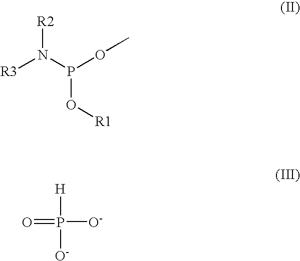

with R2 and R3 independently of one another being alkyl, where alkyl is a branched or unbranched C₁ to C₅ radical, preferably an isopropyl, and R1 is methyl, allyl (—CH₂—CH═CH₂) or preferably β-cyanoethyl (—CH₂—CH₂—CN).

and wherein

V is a branching unit with at least three binding partners, for example an atom or an atom group, preferably a nitrogen atom, carbon atom or a phenyl ring

and wherein A is an acetal of the formula (IV),

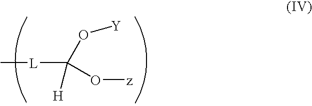

where Y and Z independently of one another are identical or different branched or unbranched, saturated or unsaturated, where appropriate cyclic, C₁ to C₁₈ hydrocarbons, preferably methyl, ethyl, n-propyl, isopropyl, n-butyl, 2-butyl, tert-butyl, particularly preferably ethyl, or wherein Y and Z together [lacuna] a radical of the structure (V) or (VI), where R4 independently of one another is identical or different and is H, methyl, phenyl, a branched or unbranched saturated or unsaturated, where appropriate cyclic, C₁ to C₁₈ hydrocarbon or a radical of the structure (VII), with R5 being identical or different and being H, methyl, alkyl, O-methyl, O-alkyl, or alkyl, where alkyl is a branched or unbranched, saturated or unsaturated, where appropriate cyclic, C₁ to C₁₈ hydrocarbon

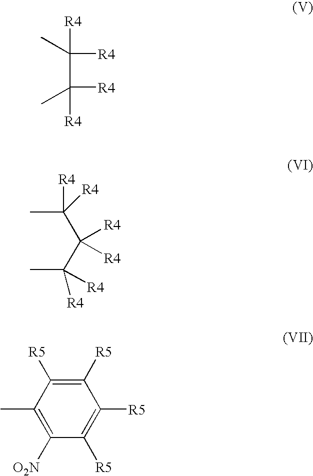

and wherein

are linkers which are suitable for linking X to A or X to V and V to A, for example branched or unbranched, saturated or unsaturated, where appropriate cyclic, C₁ to C₁₈ hydrocarbons such as, for example, Alkyl-(CₙH₂ₙ)— where n is an integer from 0 to 18, preferably 3 to 8, or is a polyether —(CH₂)ₖ—[O—(CH₂)ₘ]ₒ—O—(CH₂)ₚ— where k, m, p independently of one another are an integer from 0 to 4, preferably 2, and o is an integer from 0 to 8, preferably 2 to 4, or is an amine —(CH₂)_{w}—NH—(CH₂)ᵤ— where w and u independently of one another are an integer from 0 to 18, preferably 3 to 6, or is an amide —(CH₂)_{q}—C(O)—N—(CH₂)ᵣ— or —(CH₂)_{q}—N—C(O)—CH₂)ᵣ— where q and r independently of one another are an integer from 0 to 18, preferably 1 to 5. In this connection, the linkers L can be linked to the branching unit V via oxygen atoms.

Individual preferred examples of reactive monomers of this kind are:

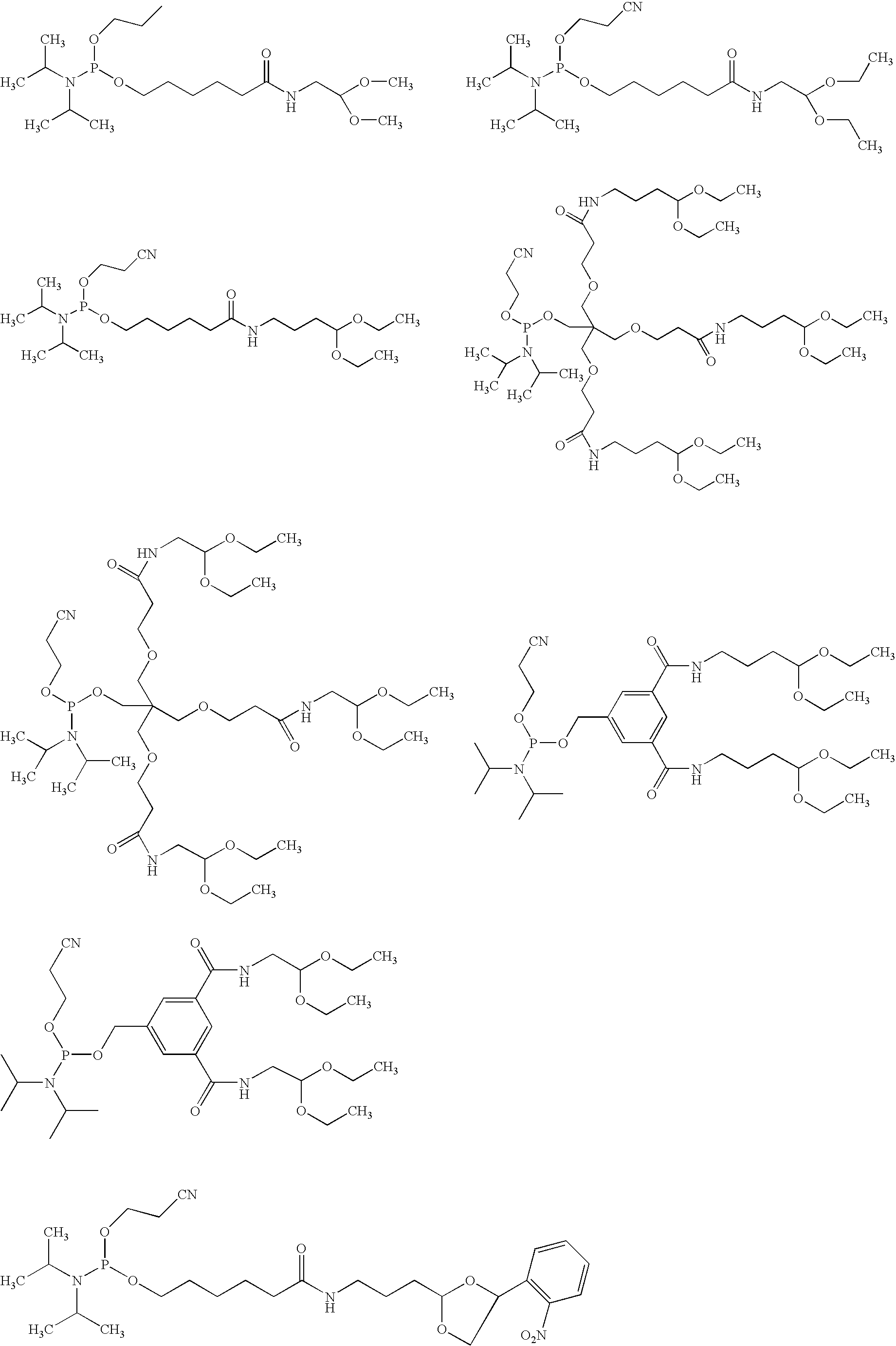

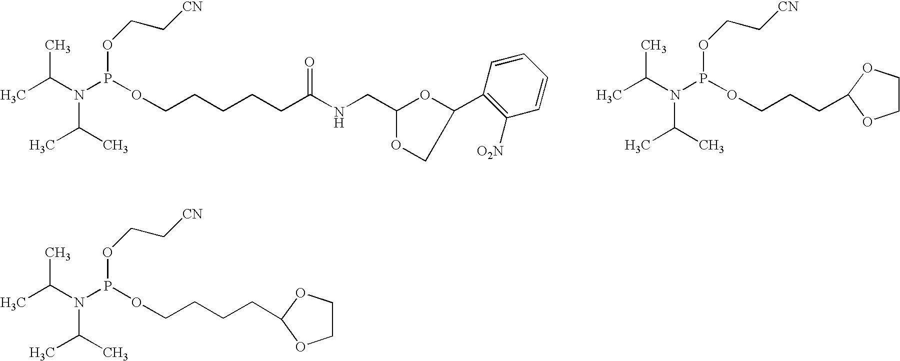

The invention further relates to mono-, oligo- and polynucleotides of any sequence, which have been modified with at least one acetal group.

Preference is especially given to mono-, oligo- and polynucleotides which are obtainable by using at least one inventive reactive monomer of the formula (I).

Examples which are obtainable are substances of the formula VIII which have a random sequence and which have been modified with at least one acetal group,

(M)ₛ[—X′—Lₗ Vᵥ(A)ₐ]_{z} (VIII)

where (M)ₛ are s monomeric units linked to one another, with s being 1 or greater, X′ is a phosphorus-containing group of the formula (IX)

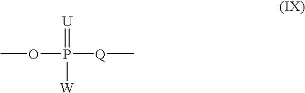

where U is O or S, W is OH, SH or H and Q is O or NH, and z is 1 or greater and l, v, a, L, V and A have the abovementioned meaning.

Linking the reactive monomers of the invention to the mono-, oligo- or polynucleotide preferably via phosphodiester, H-phosphonate, phosphorothioate, phosphorodithioate or phosphoroamidate groups of the X′ forms

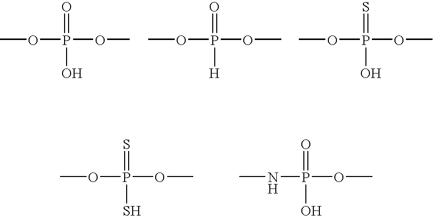

In this connection, it is possible to attach the reactive monomer of the formula (I) specifically at the terminus. Thus, z depends on the degree of branching of the nucleotide chain and is preferably between 1 and 10 and is particularly preferably 1 or 2. An additional advantage of the invention is the possibility of attaching a reactive monomer selectively to the 3′ and/or 5′ end of a DNA or RNA oligonucleotide or DNA or RNA polynucleotide or to the 2′ and/or 4′ end of a p-DNA or p-RNA oligonucleotide or p-DNA or p-RNA polynucleotide. In contrast to this, free diol groups are completely oxidized in the reaction with periodate.

Valid oligonucleotides or polynucleotides are all naturally occurring or else synthesized polymers which are capable of molecular recognition or pairing and have a repetitive structure which involves mainly phosphoric acid diester bridges. Said molecular recognition or pairing is characterized by being selective, stable and reversible and by the fact that it can be influenced, for example, by temperature, pH and concentration. For example, the molecular recognition is achieved, albeit not exclusively, by purine and pyrimidine base pairing according to the Watson-Crick rules. Examples of naturally occurring nucleotide chains are DNA, cDNA and RNA, in which nucleosides comprising 2-deoxy-D-ribose or D-ribose are linked to N-glycosidically linked heterocyclic bases via phosphoric acid diesters. Preferred examples of non-natural oligo- and polynucleotides are the chemically modified derivatives of DNA, cDNA and RNA, such as, for example, phosphorothioates, phosphorodithioates, methylphosphonates, 2′-O-methyl-RNA, 2′-O-allyl-RNA, 2′-fluoro-RNA, LNA thereof or those molecules which can pair with DNA and RNA, like PNA (Sanghivi, Y. S., Cook, D. P., Carbohydrate Modification in Antisense Research, American Chemical Society, Washington 1994) or else those molecules which, like p-RNA, homo DNA, p-DNA, CNA (DE 19741715, DE 19837387 and WO 97/43232) for example, which are capable of a molecular recognition via specific pairing properties.

The chain length range, including a monomeric building block as claimed in claim 1, is preferably from 2 to 10 000 monomeric units, and chain lengths of from 5 to 30 monomeric units are particularly preferred.

Suitable monomeric units which can be used for preparing the oligo- or polynucleotides are especially naturally occurring nucleotides, such as deoxyribonucleotides or ribonucleotides. However, it is also possible to use synthetic nucleotides which do not occur naturally.

Preferred examples of synthetic monomeric units are 2′-deoxyribofuranosylnucleotides, ribofuranoslynucleosides, 2′-deoxy-2′-flouroribofuranosylnucleosides, 2′-O-methylribofuranosylnuceosides, pentopyranosylnucleotides, 3′-deoxypentopyranosylnucleotides. Suitable heterocyclic bases for these nucleotides are inter alia: purine, 2,6-piaminopurine, 6-purinethiol, pyridine, pyrimidine, adenosine, guanosine, isoguanosine, 6-thioguanosine, xanthine, hypoxanthine, thymidine, cytosine, isocytosine, indole, tryptamine, N-phthaloyltryptamine, uracil, coffeine, theobromine, theophylline, benzotriazole or acridine and also derivatives of said heterocycles, which carry further covalently linked functional groups.

It is likewise possible to use also other monomeric units such as natural and non-natural amino acids, PNA monomers and CNA monomers.

Oligo- and polynucleotides in accordance with this invention also include those molecules which contain, in addition to the units required for molecular recognition, further molecular parts which serve other purposes such as, for example, detection, conjugation with other molecular units, immobilization on surfaces or on other polymers, spacing or branching of the nucleotide chain. They mean in particular the covalent or stably noncovalent conjugates of oligonucleotides with fluorescent dyes, chemoluminescent molecules, peptides, proteins, antibodies, aptamers, organic and inorganic molecules and also conjugates of two or more pairing systems which have different pairing modes, such as p-RNA conjugated with DNA or chemically modified derivatives thereof, p-RNA conjugated with RNA or chemically modified derivatives thereof, p-DNA conjugated with DNA or chemically modified derivatives thereof, p-DNA conjugated with RNA or chemically modified derivatives thereof, CNA conjugated with DNA or chemically modified derivatives thereof, CNA conjugated with RNA or chemically modified derivatives thereof. However, the immobilization on support surfaces such as, for example, glass, silicon, plastic, gold or platinum are of very particular interest. The surfaces in turn may contain one or more layers of coatings, preferably polymeric coatings such as polylysine, agarose or polyacrylamide. The coating may contain a plurality of staggered layers or else unarranged layers. In this connection, the individual layers may be in the form of monomolecular layers.

With respect to the present invention, conjugation means the covalent or noncovalent linkage of components such as molecules, oligo- or polynucleotides, supramolecular complexes or polymers with one or more other, different or identical components such that they form a stable unit, a conjugate, under the conditions required for their use. In this connection, the conjugation need not necessarily be covalent but can also be carried out via supramolecular forces such as van der Waals interactions, dipole interactions, in particular hydrogen bonds, or ionic interactions.

Of particular interest are furthermore conjugates with organic or inorganic molecules which possess a biological activity.

Molecules which may be mentioned in this connection are pharmaceuticals, crop protecting agents, complexing agents, redox systems, ferrocene derivatives, reporter groups, radio isotopes, steroids, phosphates, triphosphates, nucleoside triphosphates, derivatives of leading structures, transition state analogs, lipids, heterocycles, in particular nitrogen heterocycles, saccharides, branched or unbranched oligo- or polysaccharides, glycoproteins, glycopeptides, receptors or functional parts thereof such as the extracellular domain of a membrane-bound receptor, metabolites, messengers, substances which are produced in a human or animal organism in the case of pathological changes, antibodies or functional parts thereof such as, for example Fv fragments, single-chain Fv fragments or Fab fragments, enzymes, filament components, viruses, viral components such as capsids, viroids, and derivatives thereof such as, for example, acetates, substance libraries such as ensembles of structurally different compounds, preferably oligomeric or polymeric peptides, peptidoids, saccharides, nucleic acids, esters, acetals or monomers such as heterocycles, lipids, steroids or structures on which pharmaceuticals act, preferably pharmaceutical receptors, ion channels, in particular voltage-dependent ion channels, transporters, enzymes or biosynthesis units of micoorganisms.

The invention likewise relates to the aldehyde-modified p-RNA and p-DNA oligonucleotides and p-RNA and p-DNA polynucleotides which can be prepared readily from the particular acetal, for example by means of aqueous acids or photochemically.

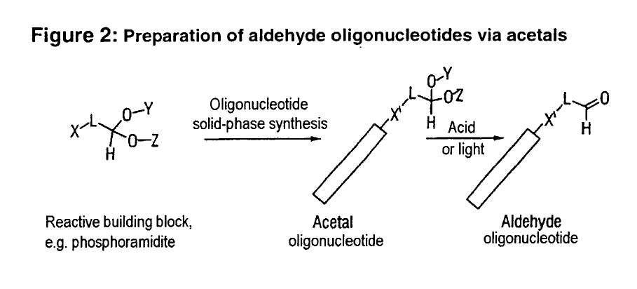

The preparation of acetal oligonucleotides or polynucleotides is effected using acetals of the formula (I) as starting material. It is possible, by way of example, to use conventional phosphoramidites which carry one or more acetal groups. These may be integrated into the oligo- or polynucleotides via the standard methods of solid-phase synthesis (FIG. 2 shows a diagrammatic representation of this).

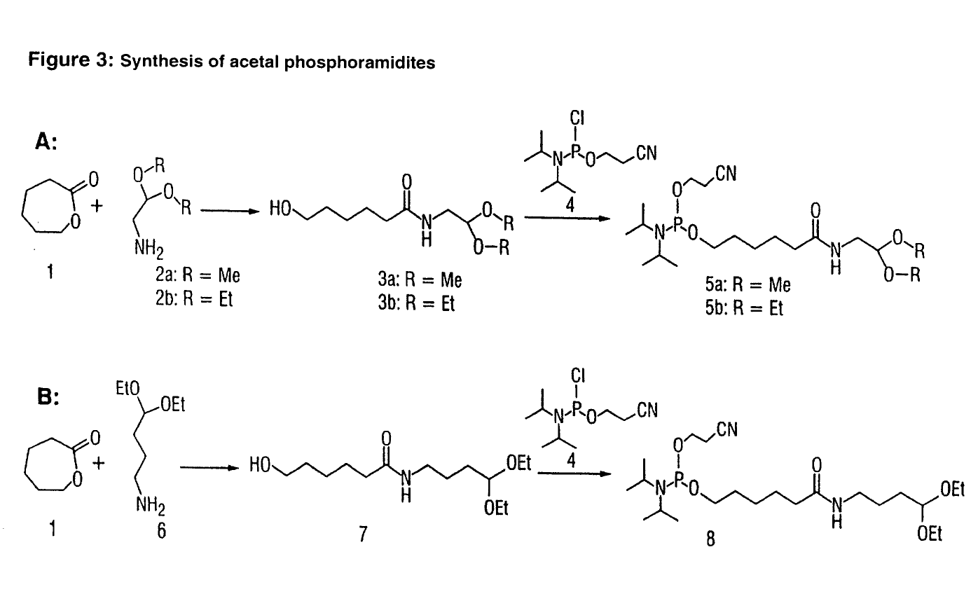

Such acetal group-carrying reactive monomeric building blocks are synthesized, for example, by reacting aminoacetals (2a, 2b, 6) (FIG. 3) with caprolactone (as described, for example, in Zhang, J.; Yergey, A.; Kowalak, J.; Kovac, P., Tetrahedron 54 (1998) 11783). The hydroxyacetals obtained, 3a, 3b or 7 are then converted into the reactive monomer for the oligonucleotide synthesis by reaction with an appropriate phosphorus reagent (as an example of this, see: I. Beaucage, S. L., Iyer, R. P., Tetrahederon 49 (1993).

As an alternative, it is possible to prepare appropriate hydroxyacetals from the halides thereof by Finkelstein's reaction or from a hydroxyaldehyde and an alcohol component by acetalization. Conversion into the reactive form is then carried out again by reaction with the corresponding phosphorus reagent.

Of particular interest are also cyclic acetals which carry an o-nitrophenyl group, since these can be converted into the aldehyde not only by acids but also by illumination with light.

The acetals are then incorporated into oligonucleotides according to the standard methods of oligonucleotide solid-phase synthesis (Beaucage, S. L.; lyer, R. P., Tetrahederon 49 (1993) 6123; Caruthers, M. H., Barone, A. D.; Beaucage, S. L.; Dodds, D. R.; Fisher, E. F.; McBride, L. J.; Matteucci, M.; Stabinksy, Z.; Tang, J. Y., Methods Enzymol. 154 (1987) 287; Caruthers M. H.; Beaton, G.; Wu, J. V.; Wiesler, W., Methods Enzymol. 211 (1992) 3).

Acetals are inert to all reaction conditions of the common oligonucleotide synthesis methods such as, for example, the phosphoramidite method.

Thus, for example, the acetals are inert to activation with tetrazole, benzylthiotetrazole, pyridinium hydrochloride, etc., capping with acetic anhydride and N-methylimidazole, oxidation, for example with iodine/water. They are likewise inert to the reaction conditions of the H-phosphonate method, such as activation with pivaloyl chloride.

Furthermore, acetals are stable to the basic reaction conditions for oligonucleotide deprotection. They withstand the customarily used concentrated aqueous ammonia solution (55° C., 2-10 h) undamaged and are not attacked by alternative reagents as used in particular cases (ethylene-diamine, methylamine, hydrazine) either (Hogrefe, R. I.; Vghefi, M. M.; Reynolds, M. A.; Young, K. M.; Arnold, L. J. Jr., Nucleic Acids Res. 21 (1993) 2031).

The aldehyde functionality is readily released from the acetals (as, for example, in Examples 8-11) by treating the acetal oligonucleotides with aqueous acids (acetic acid, trifluoroacetic acid, hydrochloric acid, etc.) or by illumination with light (for this, see also the diagrammatic representation in FIG. 2). In both cases, it is not necessary to remove the aldehyde oligonucleotide from reagents such as sodium periodate. It is sufficient, but not always necessary, to neutralize the acid. If the salt content due to neutralization of the acid is to interfere with the conversion of the aldehyde, it may also be removed via common methods such as, for example, gel filtration, dialysis, reverse-phase extraction.

The aldehyde oligo- or polynucleotides obtained in this way may be used in all linking reactions described in the literature (e.g. in Hermanson, G. T., Bioconjugate Techniques, Academic Press, San Diego 1996; Timofeev, E. N.; Kochetkova, S. V.; Mirzabekov, A. D.; Florentiev, V. L., Nucleic Acids Res. 24 (1996) 3142). The conjugation of oligo- or polynucleotides with proteins and peptides, fluorescent dyes, other oligonucleotides and the immobilization of oligo- or polynucleotides on surfaces and on other polymers are of particular interest.

Furthermore, aldehyde-modified oligo- or polynucleotides make it possible to use the reaction depicted in FIG. 1C for conjugation with peptides, proteins or other organic or inorganic molecules which carry a cystein at their N terminus. In this case, a thiazolidine derivative is formed which, with a given constitution of the aldehyde, can still be rearranged (Lemieux, G. A.; Bertozzi, C. R., Trends in Biotechnology 16 (1998) 506; Liu, C.-F.; Rao, C.; Tam, J. P., J. Am. Chem. Soc. 118 (1996) 307). This reaction has the advantage of taking place at low reactant concentrations and pH values.

The use of acetals as protective groups for aldehydes furthermore allows a particularly simple method for conjugating oligo- or polynucleotides: conjugation on the support.

To this end, the still completely or partially protected acetal oligonucleotide or acetal polynucleotide which is still immobilized on the support material of the oligonucleotide solid-phase synthesis is converted into the corresponding aldehyde oligonucleotide or aldehyde polynucleotide. It is crucial that this reaction which is made possible by aqueous acids or by illumination with light does not lead to the removal of the oligo- or polynucleotide from the support material. The support-bound aldehyde-nucleotide chain is then reacted with an appropriate reaction partner (as an example thereof, see FIG. 1). Subsequently, the oligo- or polynucleotide conjugate is removed from the support by aqueous ammonia or alternative reagents (e.g. ethylenediamine, methylamine, hydrazine) and freed of the remaining protective groups, in the case of DNA, for example, the benzoyl and isobutyryl protective groups on the exocyclic amino groups of the bases. A precondition is that the linkage formed during conjugation is stable to said deprotection conditions, which is the case for the products described by way of example in FIG. 1. This conjugation of support-bound oligo- or polynucleotides has the advantage that the excesses of the components to be conjugated and other reagents such as, for example, the reducing agent can be removed from the support-bound conjugate by simple washing. Thus it is also possible to obtain conjugates of oligo- or polynucleotides with molecules which are not accessible by direct oligonucleotide solid-phase synthesis due to specific instabilities.

### EXEMPLARY EMBODIMENTS

General Preliminary Remarks:

Unless stated otherwise, reagents from Aldrich and solvents from Riedel (p.a.) were used. Thin-layer chromatography (TLC) was carried out on plates containing silica gel 60 F254 (Merck). Column-chromatographic separations were carried out on silica gel 60 (Merck, 230-400 mesh). 1H-NMR spectra were measured at 400 MHz in a Bruker DRX 400 spectrometer and the chemical shifts were indicated as δ values against tetramethylsilane (TMS). IR spectra were measured in a Perkin Elmer Paragon 1000 FT-IR spectrometer with a Graseby Specac 10500 ATR unit. DNA oligonucleotides were prepared according to the phosphoramidite method in a PE Biosystems Expedite 8905. Acetal phosphoramidites as well as the DNA amidites were used as 0.1 M solution in dry acetonitrile. The coupling was carried out using tetrazole as activator. For p-RNA oligonucleotides, the previously described synthesis conditions were used (DE 19741715). Electrospray mass spectra (ESI-MS) were recorded in a Finnigan LCQ instrument in negative ionization mode.

The numbering indicated of the individual substances refers to the digits used in FIGS. 3 to 5.

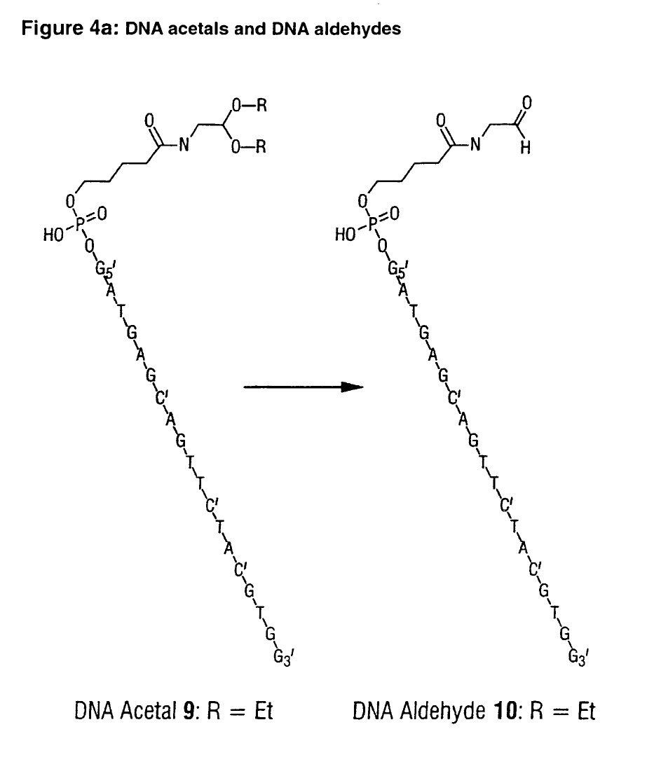

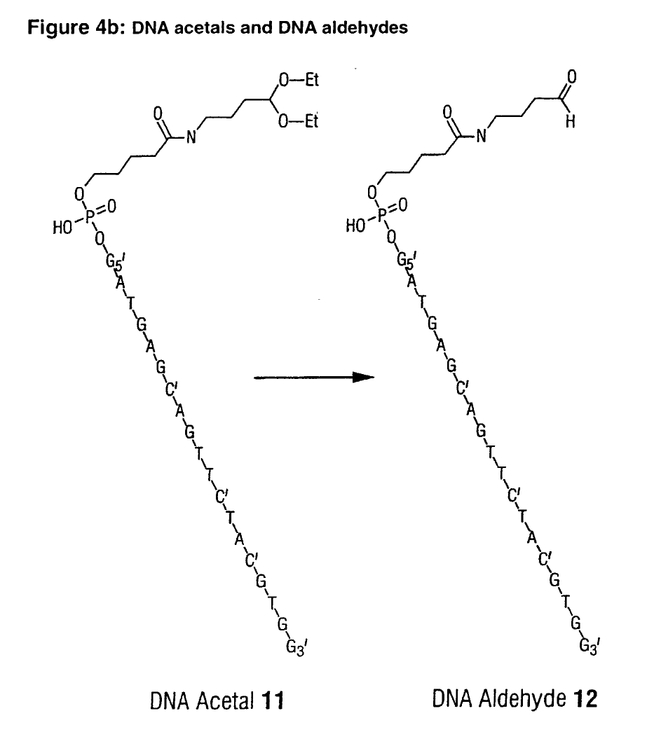

FIG. 3 describes by way of example the synthesis of acetal phosphoramidites, FIG. 4 shows examples of DNA acetals and DNA aldehydes, and Fig. [lacuna] shows examples of p-RNA acetals and p-RNA aldehydes.

Synthesis of Reactive Monomers

### Example 1

Synthesis of N-(2,2-dimethoxyethyl)-6-O-[(2-cyanoethyl)-N,N-diisopropylamidophosphoramidite]-hexamide 5a:

2.19 g (10 mmol, [219.28]) N-(2,2-dimethoxyethyl)-6-hydroxyhexamide 3a are dissolved together with 5.17 g (40 mmol, 4 eq., [129.25]) N-ethyl-diisopropylamine (Hünigs Base) in 40 ml of dry dichloromethane. 2.6 g (11 mmol, 1.1 eq., [236.68]) mono(2-cyanoethyl) N,N-diisopropylchlorophosphoramidite 4 are added dropwise over 15 min. After 1 hour, the TLC (ethyl acetate/n-heptane 2:1) indicates complete conversion. The solvent is stripped off in a rotary evaporator and the residue is applied directly to a chromatography column. Elution with ethyl acetate/n-heptane (2:1) containing a few drops of triethylamine results in 2.48 g (59%) of compound 5a as colorless oil (C₁₉H₃₈N₃O₅P; [419.51]). ¹H-NMR (CDCl₃; 400 MHZ): δ=5.71 [b, 1 H, N—H), 4.37 (t, 1 H, J=5.4 Hz, C—H), 3.89-3.67 (m, 2 H, CH₂ cyanoethyl), 3.66-3.54 (m, 4 H, CH₂, C—H i-Pr), 3.45-3.38 (m, 8 H, CH₃, CH₂), 2.64 (t, 2 H, J=6.6 Hz, CH₂), 2.19 (t, 2 H, J=7.25 Hz, CH₂), 1.77-1.59 (m, 4 H, CH₂), 1.44-1.36 (m, 2 H, CH₂), 1.19-1.16 (m, 12 H, CH₃ i-Pr); ³¹ P-NMR (CDCl₃): δ=148.0

### Example 2

N-(2,2-diethoxyethyl)-6-O-[(2-cyanoethyl)-N,N-diisopropylamidophosphoramidite]-hexamide 5b:

2.47 g (10 mmol, [247.34]) N-(2,2-diethoxyethyl)-6-hydroxyhexamide 3b are dissolved together with 5.17 g (40 mmol, 4 eq., [129.25]) N-ethyl-diisopropylamine (Hünigs Base) in 40 ml of dry dichloromethane. 2.6 g (11 mmol, 1.1 eq., [236.68]) mono(2-cyanoethyl) N,N-diisopropylchlorophosphoramidite 4 dissolved in 5 ml of dichloromethane are added dropwise over 30 min. After another 30 min, the TLC (ethyl acetate/n-heptane 2:1) indicates complete conversion. The solvent is stripped off in a rotary evaporator and the residue is taken up in ethyl acetate/n-heptane (2:3). The precipitated hydrochloride is filtered off by suction and the filtrate is applied directly to a chromatography column. Elution with ethyl acetate/n-heptane (1:1) containing a few drops of triethylamine results in 2.96 g (66%) of compound 5b as colorless oil (C₂₁H₄₂N₃O₅P; [419.51]). ¹H-NMR (CDCl₃; 400 MHZ): δ=5.72 [b, 1 H, N—H), 4.49 (t, 1 H, J=5.4 Hz, C—H), 3.89-3.50 (m, 10 H, 2×CH₂, CH₃, C—H i-Pr), 3.38 (t, 2 H, J=5.64 Hz, CH₂), 2.64 (t, 2 H, J=5.9 Hz, CH₂), 2.19 (t, 2 H, J=7.52 Hz, CH₂), 1.68-1.59 (m, 4 H, CH₂), 1.44-1.38 (m, 2 H, CH₂), 1.23-1.16 (m, 18 H, CH₃ i-Pr, CH₃ Et); ³¹P-NMR (CDCl₃): δ=148.0

### Example 3

N-(2,2-diethoxybutyl)-6-O-[(2-cyanoethyl)-N,N-diisopropylamidophosphoramidite]-hexamide 8:

1.75 g (6.35 mmol, [275.39]) N-(2,2-diethoxybutyl)-6-hydroxyhexamide 7 are dissolved together with 1.64 g (12.7 mmol, 4 eq., [129.25]) N-ethyldiisopropylamine (Hünigs Base) in 30 ml of dry dichloromethane. 1.65 g (6.99 mmol, 1.1 eq., [236.68]) mono(2-cyanoethyl) N,N-diisopropylchlorophosphoramidite 4 dissolved in 2 ml of dichloromethane are added dropwise over 40 min. After another 30 min, the TLC (ethyl acetate/n-heptane 10:1) indicates complete consumption of the reactant. The reaction is stopped with methynol and the solvent is stripped off in a rotary evaporator. The residue is applied directly to a chromatography column. Elution with ethyl acetate/n-heptane (10:1) containing a few drops of triethylamine results in 1.87 g (62%) of compound 8 as colorless oil (C₂₃H₄₆N₃O₅P; [475.61]). ¹H-NMR (CDCl₃; 400 MHZ): δ=5.74 [b, 1 H, N—H), 4.48 (t, 1 H, J=5.1 Hz, C—H), 3.88-3.76 (m, 2 H), 3.69-3.45 (m, 8 H), 3.26 (q, 2 H, J=6.72 Hz, CH₂), 2.64 (t, 2 H, J=6.45 Hz, CH₂), 2.16 (t, 2 H, J=7.25 Hz, CH₂), 1.69-1.56 (m, 8 H, CH₂), 1.43-1.37 (m, 2 H, CH₂), 1.22-1.16 (m, 18 H, CH₃ i-Pr, CH₃ Et); ³¹P-NMR (CDCl₃): δ=148.0

Synthesis of Acetal- and Aldehyde-Modified Oligonucleotides:

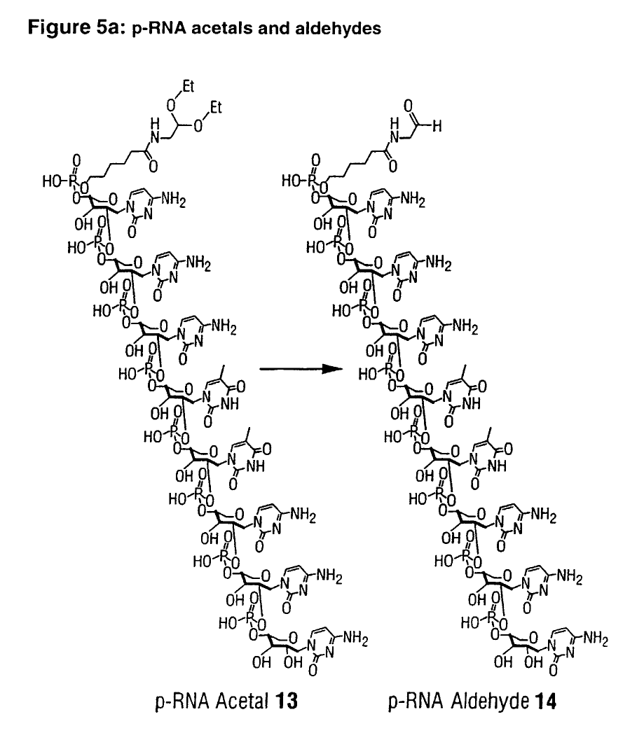

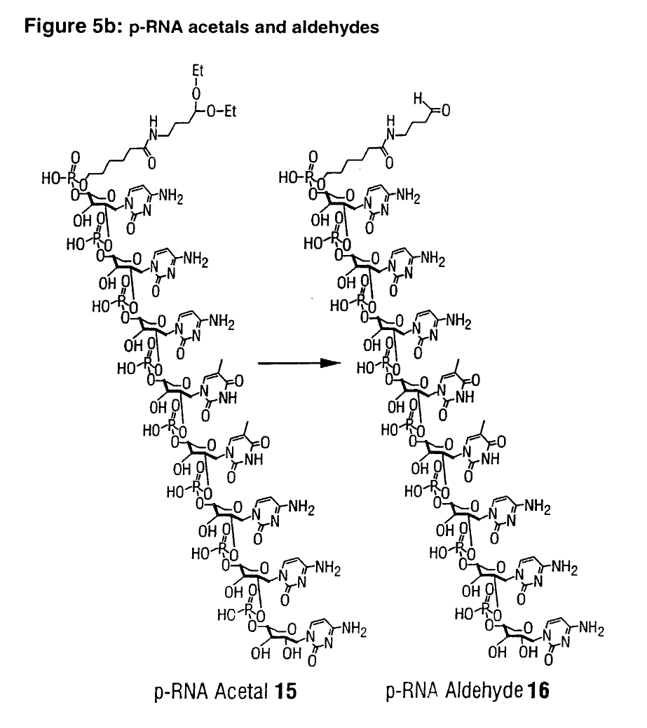

The introduction of aldehydes via acetals is shown both for DNA and p-RNA oligonucleotides. FIGS. 4 and 5 show the sequences of the oligonucleotide examples.

### Example 4

DNA Acetal 9 from Diethylacetal 5b (K3194/3196 O4)

The oligonucleotide synthesis is carried out on the 1 μmol scale according to the protocols provided by the manufacturer of the instrument. A 0.1 M solution of the phosphoramidite 5b is coupled as the last monomer under the standard conditions. The support-bound oligonucleotide is removed and deprotected by treatment with an aqueous 25% ammonia solution at 80° C. for 10 h. After removing the support, the solution is concentrated under reduced pressure and the residue is dissolved in water. The oligonucleotide is purified via RP-HPLC. Column: Merck LiChrospher RP 18, 10 μM, analytical: 4×250 mm, flow-rate=1.0 ml/min, semipreparative: 10×250, flow rate=3.0 ml/min; buffer: A: 0.1 M triethylammonium acetate (TEAA) pH=7.0 in water, B: 0.1 M TEAA pH=7.0 in acetonitrile/water (95:5); gradient: 0% B to 100% B in 100 min for analytical and preparative separations). Retention time DNA acetal 9: 22.8 min; MS: calc.: [6193], obs.: [6195]

### Example 5

DNA Acetal 11 from Diethylacetal 8 (K3208/3214/3218 O16)

The oligonucleotide synthesis and workup are carried out as described in Example 4. Retention time DNA acetal 11: 23.4 min; MS: calc.: [6222], obs.: [6221]

### Example 6

p-RNA Acetal 13 from Diethylacetal 5b (K3168 O16)

The oligonucleotide synthesis is carried out as described in Example 4. Deviating from this protocol, a longer coupling time and the activator pyridinium hydrochloride were used for p-RNA. In this case, the acetal phosphoramidites are also coupled using pyridinium hydrochloride as activator. First, a 1.5% (w/v) solution of diethylamine in dichloromethane is added to the support and the mixture is incubated with shaking in the dark at room temperature overnight (15 h). The solution is discarded and the support is washed with in each case three portions of the following solvents: CH₂Cl₂, acetone, water. The p-RNA is then removed from the CPG support and deprotected by treatment with aqueous 24% hydrazine hydrate at 4° C. for 18 h. Hydrazine is removed by solid-phase extraction using Sep-Pak C18 cartridges (0.5 g Waters, No. 20515; activation with 10 ml of acetonitrile, binding of the hydrazine solution diluted with the fivefold volume of triethylammonium bicarbonate buffer (TEAB) pH 7.0, washing with TEAB and elution of the oligonucleotide with TEAB/acetonitrile (1:2)). Oligonucleotide-containing fractions are combined and concentrated to dryness under reduced pressure. The analysis and preparative purification are carried out via RP-HPLC, as described in Example 4. Retention time DNA acetal 13: 22.0 min; MS: calc.: [2719], obs. [2718]

### Example 7

p-RNA Acetal 15 from Diethylacetal 8 (K3208/3214/3218 O16)

The oligonucleotide synthesis and workup are carried out as described in Example 6. Retention time p-RNA acetal 15: 24.0 min; MS: calc.: [2747], obs.: [2747]

Conversion of Acetal Oligonucleotides to Aldehyde Oligonucleotides:

General Protocol:

The acetal oligonucleotide is dissolved in water and admixed with an excess of aqueous acid (e.g. HCl). The oligonucleotide concentration in the reaction solution obtained in this way is usually between 20 and 60 μM, and a large excess of acid is used (up to 5×10⁴ mol equivalents). The solution is incubated at room temperature and the reaction progress is monitored via HPLC. After complete conversion of the acetal oligonucleotide, the solution is neutralized with aqueous NaOH. The aldehyde-oligonucleotide solution obtained in this way may be used directly for conjugation reactions or desalted via the usual methods such as gel filtration or solid-phase extraction (cf. Example 6).

### Example 8

DNA Aldehyde 10 from DNA Acetal 9

26 nmol of acetal 10 are admixed with 1 ml of 1 M aqueous HCl and incubated at room temperature for 6.5 h. The reaction progress can be followed by means of RP-HPLC under the conditions indicated in Example 4. The acid is neutralized by adding 1 N aqueous NaOH. The DNA-aldehyde solution obtained in this way may be used directly for conjugations or purified via RP-HPLC. Retention time DNA aldehyde 10: 20.6 min.

### Example 9

DNA Aldehyde 12 from DNA Acetal 11

120 nmol acetal 11 are reacted with 2 ml of 1 M aqueous HCl, as described in Example 8, to give DNA aldehyde 12. Retention time: 21.5 min; MS: calc.: [6148], obs.: [6147]

### Example 10

p-RNA Aldehyde 14 from DNA Acetal 13

16 nmol acetal 13 are reacted with 400 μl of 0.5 M aqueous HCl, as described in Example 8, to give DNA aldehyde 14. Retention time: 19.2 min; MS: calc.: [2645], obs.: [2645]

### Example 11

p-RNA Aldehyde 16 from DNA Acetal 15

50 nmol acetal 15 are reacted with 1 ml of 1 M aqueous HCl, as described in Example 8, to give DNA aldehyde 16. Retention time: 20.0 min; MS: calc.: [2673], obs.: [2672]

Conjugation Reactions of Aldehyde Oligonucleotides:

General Protocol A (Conjugation in Solution):

(I) 10 μL of a solution of a hydrazide or amine (5 to 20 mM) and 10 μL of a 100 mM aqueous NaCNBH₄ solution are diluted with acetate buffer (pH 5) to 500 μL. To this, 1-5 nmol of the aldehyde oligonucleotide dissolved in a few μL of water are added. After 2 h at room temperature, the solution is desalted by gel filtration and the conjugate purified via HPLC.

(II) As an alternative, the aldehyde-oligonucleotide solution obtained by neutralizing the acid (cf. 3.1.3) may be admixed with 100 mole equivalents of hydrazide or amine and 1000 mole equivalents of NaCNBH₄. The mixture is diluted with acetate buffer pH 5, if required. After 2 h at room temperature, the mixture is desalted by gel filtration and the conjugate purified via HPLC.

General Protocol B (Conjugation on Solid Phase):

First, an acetal oligonucleotide is prepared by solid-phase synthesis as described in Example 4 and Example 6. The support-bound oligonucleotide is then admixed first with a 1.5% (w/v) solution of diethylamine in dichloromethane and incubated with shaking in the dark at room at room temperature overnight (15 h). The solution is discarded and the support is washed with in each case 3 portions of the following solvents: CH₂Cl₂, acetone, water. The support-bound acetal oligonucleotide is converted into a support-bound aldehyde oligonucleotide by treating the support with a 0.1 to 1 M aqueous acid solution (e.g. HCl) at room temperature for 2 h. This is followed by washing with water until the filtrate shows a neutral pH. For conjugation, an incubation with a solution of a hydrazide or amine and NaCNBH₄ in acetate buffer is carried out with shaking at room temperature for several hours. The conjugate is then removed from the support and deprotected by treatment with hydrazine or ammonia (cf. Examples 4 and 6). Workup and purification are carried out as described in Example 6.

1. A reactive monomer of the formula (I), wherein l, v independently of one another are 0 or 1 and a is an integer between 1 and 5
X—Lₗ—Vᵥ—(A)ₐ (I)
where
X is a phosphoramidite (II), 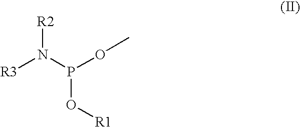
wherein R2 and R3 independently of one another are a branched or unbranched C₁ to C₅ alkyl radical and R¹ is methyl, allyl or β-cyanoethyl,
V is a branching unit composed of an atom or of a molecule having at least three binding partners,
A is an acetal of the formula (IV), 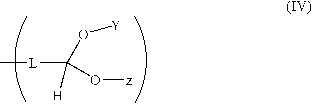
where the radicals Y and Z independently of one another are identical or different branched, unbranched or cyclic, saturated or unsaturated hydrocarbons having from one to 18 carbon atoms, it also being possible for the radicals Y and Z to be linked to one another,
and wherein
L are linkers which are suitable for linking X to A or X to V and V to A.

2. A reactive monomer as claimed in claim 1, wherein the phosphorus-containing group X is a phosphoramidite (II),
where R2 and R3 independently of one another is an isopropyl radical.

3. A reactive monomer as claimed in either of claims 1 and 2, wherein the branching unit V is a nitrogen atom, carbon atom or a phenyl ring.

4. A reactive monomer as claimed in any of the preceding claims, wherein the radicals Y and Z independently of one another are methyl, ethyl, n-propyl, isopropyl, n-butyl, 2-butyl, tert-butyl.

5. A reactive monomer as claimed in any of claims 1 to 3, wherein Y and Z together are a radical of the structure (V) or (VI) 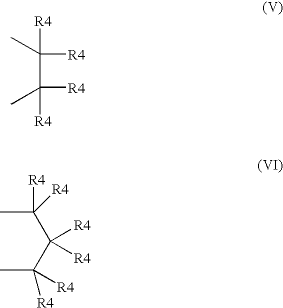
where the substituents R4 independently of one another are H, methyl, phenyl, branched, unbranched or cyclic, saturated or unsaturated C₁ to C₁₈ hydrocarbons or a radical of the structure (VII) 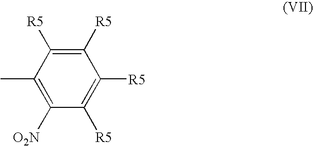
and the substituents R5 independently of one another are H, methyl, alkyl, O-methyl, O-alkyl, or alkyl, with alkyl being branched, unbranched or cyclic, saturated or unsaturated C₁ to C₁₈ hydrocarbon radicals.

6. A reactive monomer as claimed in any of the preceding claims, wherein the linkers L are selected from the group consisting of branched, unbranched or cyclic, saturated or unsaturated C₁ to C₁₈ hydrocarbons or the group which is a polyether —(CH₂)ₖ—[O—(CH₂)ₘ]ₒ—O—(CH₂)ₚ— where k, m, p independently of one another are an integer from 0 to 4, and o is an integer from 0 to 8, or the group which is an amine —(CH₂)_{w}—NH—(CH₂)ᵤ— where w and u independently of one another are an integer from 0 to 18, or the group which is an amides —(CH₂)_{q}—C(O)—N—(CH₂)ᵣ— or —(CH₂)_{q}—N—C(O)—(CH₂)ᵣ— where q and r independently of one another are an integer from 0 to 18, it also being possible for the linker L to be linked to V via an oxygen bridge.

7. A reactive monomer as claimed in any of the preceding claims, wherein the linkers L are selected from the group consisting of the (CₙH₂ₙ)-alkyl radicals where n is an integer from 0 to 18 or the group which is a polyether —(CH₂)k-[O—(CH₂)ₘ]_{o—O—(CH}₂)p- where k, m, p independently of one another are 2 and o is an integer from 2 to 4 or the group which is an amine —(CH₂)_{w}—NH—(CH₂)ᵤ— where w and u independently of one another are an integer from 3 to 6 or the group which is an amide —(CH₂)_{q}—C(O)—N—(CH₂)ᵣ— or —(CH₂)_{q}—N—C(O)—(CH₂)ᵣ— where q and r independently of one another are an integer from 1 to 5, it being possible for the linkers L to be linked to V also via an oxygen bridge.

8. A reactive monomer as claimed in one or more of the preceding claims, which has the following structure

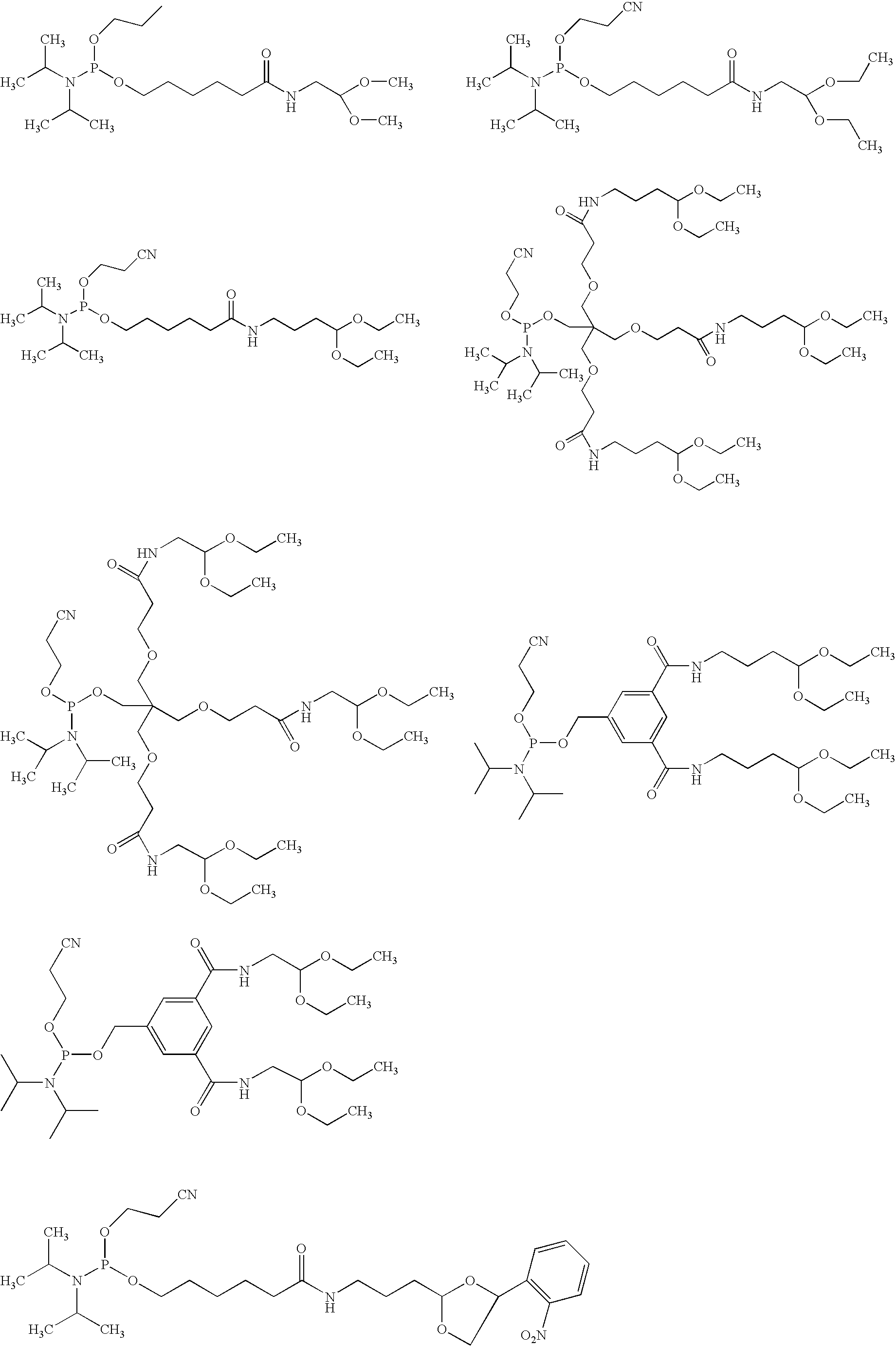

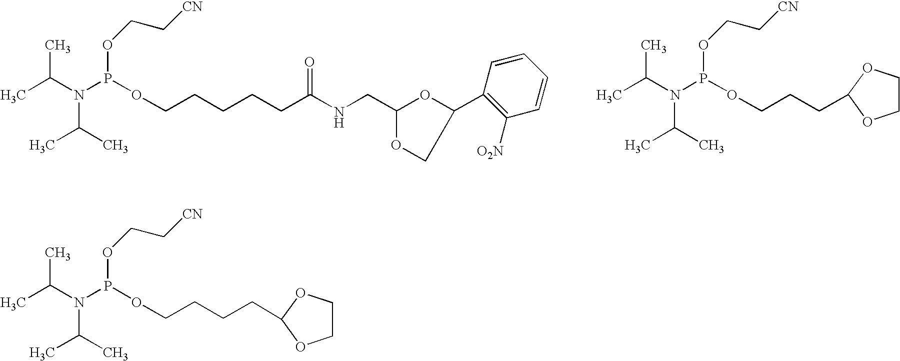

9. A mono-, oligo- or polynucleotide, obtainable by linking the mono-, oligo- or polynucleotide terminally to at least one reactive monomer of the formula (I).
X—Lₗ—Vᵥ—(A)ₐ (I)
wherein l, v independently of one another are 0 or 1 and a is an integer between 1 and 5, where
X is a phosphoramidite (II) or a phosphonate (III), 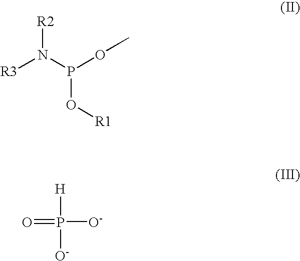
wherein R2 and R3 independently of one another are a branched or unbranched C₁ to C₅ alkyl radical and R1 is methyl, allyl or β-cyanoethyl,
V is a branching unit composed of an atom or of a molecule having at least three binding partners,
A is an acetal of the formula (IV), 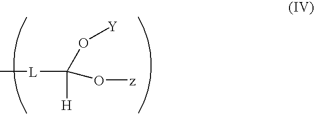
where the radicals Y and Z independently of one another are identical or different branched, unbranched or cyclic, saturated or unsaturated hydrocarbons having from one to 18 carbon atoms, it also being possible for the radicals Y and Z to be linked to one another,
and wherein
L are linkers which are suitable to link X to A or X to V and V to A. M. A mono-, oligo- or polynucleotide as claimed in claim 9, which corresponds to the formula VIII
(M)ₛ[—X′—LₗVᵥ(A)ₐ]_{z} (VIII)
where (M)ₛ are from s monomeric units of any sequence, where s is 1 or greater and (M)ₛ can be branched or unbranched, and X′ is a phosphorus-containing group of the formula (IX), which is terminally linked to the mono-, oligo- or polynucleotide, 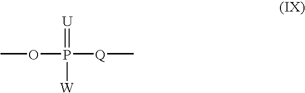
where U is O or S, W is OH, SH or H and Q is O or NH, and in which z is 1 or greater and l, v, a, L, V and A have the above-mentioned meaning.

11. A mono-, oligo- or polynucleotide as claimed in either of claims 9 or 10, comprising naturally occurring nucleotides and/or non-natural nucleotides in any sequence.

12. A mono-, oligo- or polynucleotide as claimed in any of claims 9 to 11, comprising nucleotides in the form of DNA, cDNA, RNA and/or chemically modified DNA, cDNA or RNA.

13. A mono-, oligo- or polynucleotide as claimed in any of claims 10 to 12, comprising non-natural nucleotides from the group consisting of phosphorodithioate, methyl phosphonate, 2′-O-methyl RNA, 2′-O-allyl-RNA, 2′-fluoro RNA, LNA, PNA p-RNA, homo DNA, p-DNA, CNA nucleotides.

14. A mono-, oligo- or polynucleotide as claimed in any of claims 9 to 13, wherein the chain, including a monomeric building block as claimed in claim 9, comprises 2 to 10,000 monomeric units.

15. A mono-, oligo- or polynucleotide as claimed in any of claims 9 to 13, wherein the chain, including a monomeric building block as claimed in claim 9, comprises 5 to 30 monomeric units.

16. A mono-, oligo- and polynucleotide as claimed in any of claims 9 to 15, which comprise covalently or stably noncovalently conjugated molecule parts, from the group consisting of fluorescent dyes, peptides, proteins, antibodies, polymers, aptamers, organic molecules, inorganic molecules, other oligo- or polynucleotides, and/or covalently or stably noncovalently conjugated surfaces of solid coated or uncoated support materials.

17. A mono-, oligo- and polynucleotide, which have been modified with at least one aldehyde group and in which the nucleotide chain comprises p-RNA, homo DNA, p-DNA or CNA.

18. A method for preparing aldehyde-modified oligo- or polynucleotides, comprising
a) coupling a reactive monomer of any of claims 1 to 8 to an oligonucleotide and
b) treatment with acid or light to generate the aldehyde.

19. The method as claimed in claim 18, wherein the aldehyde group(s) are subjected to a further conjugation.

20. The method as claimed in claim 18 or 19, wherein the aldehyde group(s) is subjected to a further conjugation with an amine, hydrazine or with a peptide, protein, organic molecule with terminal cystein.

21. The method as claimed in any of claims 18 to 20, wherein the preparation of the oligo- or polynucleotides is carried out completely or partially under the conditions of solid-phase oligonucleotide syntheses.

22. The use of reactive monomers as claimed in claim 1 to 8 or of mono-, oligo- or polynucleotides as claimed in claim 9 to 17 for oligo- or polynucleotide synthesis or oligo- or polynucleotide duplication.

23. The use of reactive monomers as claimed in claim 1 to 8 or of mono-, oligo- or polynucleotides as claimed in claims 9 to 17 in the phosphoramidite method or the PCR.

24. The use of mono-, oligo- or polynucleotides as claimed in claim 17 for conjugation reactions as claimed in claim 19 or 20.
